(19) United States
(12) Patent Application Publication
(10) Pub. No.: US 2006/0020157 A1
(43) Pub. Date: Jan. 26, 2006

(54) Internal radiation therapy for low lying lesions

(76) Inventor: Gary Bouldin Stillwagon, Atlanta, GA (US)

Correspondence Address: GARY B. STILLWAGON, 655 RIVER CHASE RIDGE, ATLANTA, GA, 30328, US

(21) Appl. No.: 10/899,890

(22) Filed: Jul. 26, 2004

(51) Int. Cl. A61N 5/00

(52) U.S. Cl. 600/6

(57) ABSTRACT

The present invention presents a novel approach to radiation therapy of the distal rectum. The novel idea is the combination of this device plus the treatment regimen to compress the time required to administer radiotherapy to this area to make radiotherapy more acceptable and available as well as to possibly expand the use of organ preservation surgery for disorders in this anatomic area.

The present invention relates to the field of radiation therapy of low lying bowel cancers, but has application to other areas.

There are several methods that have been applied to irradiate the distal gastrointestinal tract. These, include external beam methods as well as internal methods (brachytherapy). The latter methods are often difficult and cumbersome to administer.

In general, radiotherapy as applied to the distal gastrointestinal tract has used in pre-operative, post-operative, definitive and palliative modes.

When used in the pre-operative mode, radiotherapy can perhaps downstage cancers to attempt to make surgical resections easier.

Radiotherapy has application in the post-operative situation when the pathologic findings after surgical resection appear to warrant its consideration.

Radiation therapy has been used rather widely in the definitive mode as well. Examples would include, without limitation, anal cancers, unresectable rectal cancers and resectable recto-sigmoid cancers that occur in patients who are medically not candidates for surgery or who refuse surgery.

Palliative uses of radiation therapy includes addressing such quality of life issues as stoppage of bleeding.

All of the above mentioned variations on the theme of the use of radiation therapy can be used more advantageously with the present invention.

Well known problems with the use of external radiation therapy include the toxicity caused by bowel irradiation and the relatively long time duration over which such treatments must be given to result in a reasonable ratio of benefit versus risk.

Well known problems with the use of internal radiation therapy can relate to the difficulty in implementing the treatment and the lack of a cohesive procedure to incorporate it into an overall management program.

The above mentioned problems are largely circumvented by the present invention.

Organ preservation can sometimes be hindered by the lack of convenient availability of radiation therapy facilities or the significant time commitment needed to implement the radiotherapy course. This hindrance was shown rather clearly in breast conservation treatment in breast cancer.

Here a device is described that is used to implement an abbreviated short course of radiotherapy.

The device consists of one tube constructed inside another. A portion of the outside most tube is inflated with a substance of similar radiation characteristics to tissue, such as water, after insertion of the device into the appropriate portion of bowel needing treatment. This is intended to conform to wall of the bowel containing the tumor with a margin. The inner tube is used to connect to a separate device to introduce radioisotope(s). Next a planning session then ensues that typically involves determination of the location of the device relative to important anatomic structures and of the dose distribution produced by the radioisotope(s). Treatment can be initiated based on this planning session by introduction of radioisotope into the inner tube. Once treatment is complete, the device can be deflated and removed. Devices can be similarly placed on separate days to complete the radiotherapy course. A course can comprise one week or other duration depending on the dose distribution, treatment goal, and whether this treatment is being used in combination with other treatments.

1. The novel idea is the combination of this device plus the treatment regimen to compress the time required to administer radiotherapy to this area to make radiotherapy more acceptable and available as well as to possibly expand the use of organ preservation surgery for disorders in this anatomic area.
